(19) United States
(12) Patent Application Publication
(10) Pub. No.: US 2022/0071519 A1
(43) Pub. Date: Mar. 10, 2022

(54) REAL-TIME CONTINUOUS GLUCOSE MONITORING BASED METHOD TO TRIGGER CARBOHYDRATES ASSUMPTION TO REVENT/MITIGATE HYPOGLYCEMIC EVENTS

(71) Applicant: DexCom, Inc., San Diego, CA (US)

(72) Inventors: Giovanni SPARACINO, Padova (IT); Nunzio CAMERLINGO, Bacoli (IT); Martina VETTORETTI, Valla di Rese Pio X (IT); Andrea FACCHINETTI, Trissino (IT); Simone DEL FAVERO, Valle di Cadore (IT); Giacomo CAPPON, Padova (IT)

(21) Appl. No.: 17/291,158

(22) PCT Filed: Nov. 6, 2019
(86) PCT No.: PCT/EP2019/080444

§ 371 Date: May 4, 2021

Related U.S. Application Data
(60) Provisional application No. 62/756,227, filed on Nov. 6, 2018.

(51) Int. Cl. A61B 5/145; A61B 5/00

(52) U.S. Cl. CPC A61B 5/14532; A61B 5/746; A61B 5/7275

(57) ABSTRACT

Mitigation of the risk of prolonged hypoglycemia in T1D management requires patient to assume a small dose of fast-acting carbohydrates, called hypotreatment (HT), as soon as hypoglycemia is detected. This invention consists in a method that, on the basis of the datastream generated by a continuous glucose monitoring (CGM) sensor, triggers the assumption of preventive HTs i.e., snacks that, being quickly absorbed into the circulation, avoid, or at least mitigate, a forthcoming hypoglycemic event. The method resorts to the “dynamic risk” (DR) non-linear function, which combines current glycemia with its rate-of-change provided by CGM, adapted to distinguish the severity of the about-to-happen hypoglycemia. The method has been tested in a simulated realistic scenario. Results show that the administration of an HT in advance, as triggered by the new method, brings to a strong reduction of the time that a patient would have spent in hypoglycemia assuming the HT at hypoglycemic threshold crossing.

### FIELD OF THE INVENTION

The present invention relates to a method, based on the real-time processing of continuous glucose monitoring (CGM) data, that can trigger patients affected by diabetes to assume a small dose of fast-acting carbohydrates, to prevent, or at least mitigate, an about to happen hypoglycemic event.

### BACKGROUND OF THE INVENTION

Diabetes is a chronic disease characterized by abnormal glycemic values due to the inability of the pancreas to produce insulin (Type 1 diabetes) or to the inefficiency of insulin secretion and action (Type 2 diabetes). Patients affected by diabetes need to monitor their blood glucose (BG) level during all day, in order to control it and take countermeasures to keep it inside the safety euglycemic range of 70-180 mg/dl as much as possible. Diabetes therapy consists in diet, physical exercise and exogenous insulin and drugs administration, opportunely tuned based on glucose concentration measurements. According to the American Diabetes Association (ADA) 2018 Standards of Medical Care in Diabetes [1], when hypoglycemia is detected, individuals with diabetes should assume a small amount of fast-acting carbohydrates (also called hypotreatment—HT) in order to avoid a long permanence in hypoglycemia. ADA also suggests re-checking the glucose concentration fifteen minutes after the first treatment and, if measured glucose concentration is still in hypoglycemia, the patient should assume an additional HT.

Continuous glucose monitoring (CGM) are the state-of-the-art systems to monitor the glucose concentration in real-time, providing a glucose concentration value every 1-5 minutes for several consecutive days/weeks. Most of CGM devices are equipped with an alert generator system, which allows detecting in real-time hyperglycemia (e.g., CGM>180 mg/dl) and hypoglycemia (e.g., CGM<70 mg/dl). When hypoglycemic and hyperglycemic thresholds are crossed, the CGM sensors can generate a visual/acoustic alert for the user, suggesting them taking HT and insulin correction boluses, respectively. Focusing on hypoglycemia, if the HT is administered when the hypoglycemic threshold is crossed, hypoglycemia cannot be avoided, since carbohydrates take time to reach the blood stream and rise the glucose concentration. The high sampling frequency of CGM measurements and their real-time availability allows CGM systems to capture in real-time the dynamics of glucose concentration that can be exploited to make short-term predictions of future glucose concentration (e.g., 20-30 minutes ahead of time). If hypoglycemia is predicted, a preventive hypoglycemic alert can be provided to the CGM user who can make a suitable treatment decision (e.g., to assume an HT) to mitigate, or avoid at all, the incoming hypoglycemic event.

In this document, we describe a method to trigger the assumption of carbohydrates to prevent and treat hypoglycemia, based on CGM data, and specifically a method to predict forthcoming hypoglycemia, estimate its severity, calculate the clinical risk associated to the current glucose concentration value, and, finally, trigger HT administration accordingly.

### PRIOR ART

Concerning the use of real-time CGM sensor data, there are not any explicit contribution on strategies for the assumption of preventive HTs to avoid/mitigate a predicted hypoglycemic episode.

In general, it is worth noting the following methods to handle hypoglycemia:
Greenburg et al. [2] (W.O. Patent 2014/140062 A2, Sep. 18, 2014) proposed a method to handle hypoglycemia based on the computation of a carbohydrate dose, depending only on the current glucose concentration value. The method does not provide any prediction of the incoming hypoglycemia and its severity and does not exploit any clinical risk function for such a purpose.
Mintz et al. [3] (U.S. Patent 2009/0300398 A1, Jan. 29, 2009) proposed a method for glucose control, which includes a strategy to suggest the patient to assume carbohydrates in case the measured glucose value is below the glucose target. The method is designed for any device to measure blood glucose, so it does not exploit the possibility to use CGM data to predict hypoglycemia and its severity in advance, and it does not exploit any clinical risk function for its purpose.
Budiman et al. [4] (U.S. Patent 2014/0121488 A1, May 1, 2014) proposed a Kalman filter-based method to generate glycemic alarms, suggesting several countermeasures for hypoglycemia, including the assumption of carbohydrates. Differently from the present invention, the method performs a punctual prediction and does not take into account the clinical risk associated to the current glucose concentration value. Moreover, no explicit protocols (including HT administration time, dose of each HT and re-treats) are proposed to trigger an HT assumption, which is the main aim of the present patent application.

For completeness, it is also important to point out that:
for insulin pump users only, hypoglycemia can be mitigated by reducing/suspending the basal insulin infusion. For example, Hughes et al. [5] (J Diabetes Sci Technol 2010) developed two methods for such a purpose: 1) “Brakes”, which exploits CGM data to compute an attenuation factor for basal insulin release; 2) “Power Brakes”, in which the attenuation factor is calculated based on predicted glucose concentration obtained by a state model describing the glucose-insulin system and the Kalman filter. The methods, patented by Kovatchev et al. [6] (U.S. Patent 2012/0059353 A1, Mar. 8, 2012), use the clinical risk concept to compute the attenuation factor. Patek et al. [7] (IEEE Trans Biomed Eng 2012) proposed a method for basal insulin attenuation based on a linear predictor to estimate glucose trend and a system to correct glucose predictions for insulin-on-board. Differently from the present invention, despite using the concept of clinical risk, all these methods work on the modulation/suspension of basal insulin infusion and not on triggering preventive HTs.
several methods for CGM-based prediction of future hypoglycemia have been developed, but none of them suggested a method to treat the predicted hypoglycemia.

Finally, Sparacino et al. [8] (U.S. Patent 2013/0109944 A1, May 2, 2013) proposed the concept of Dynamic Risk (DR) and its usage to allow CGM users to visualize the clinical risk. The DR, that in this invention we employ in the preferred embodiment, was not used to trigger HTs for the prevention, mitigation, and treatment of hypoglycemia, as in the aim of the present patent application.

### SUMMARY OF THE INVENTION

The present invention relates to a method to trigger carbohydrates assumptions to avoid, or at least mitigate, hypoglycemic events, based on CGM data, and particularly to a method to predict the forthcoming hypoglycemic event, estimate its severity, and trigger HT administrations.

The method receives in input the outputs of the real-time CGM device i.e., current CGM value and current estimated glucose rate-of-change (ROC). Based on these two values, the algorithm computes the clinical risk at current time, forecasts if a hypoglycemic event is about to happen, and classify the upcoming episode in “Class A”, if glucose concentration is predicted to rapidly drop in hypoglycemia, or in “Class B” otherwise. In a preferred embodiment, the clinical risk is quantified by the dynamic risk (DR) function [9] (Diabetes Technol Ther 2012). Then, different HTs are triggered for “Class A” and “Class B” predicted events. After the first HT administration, the method periodically re-checks for the conditions and eventually triggers the ingestion of other HTs.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a flowchart representing how the method works.

FIG. 2 is a graph showing how the proposed method works for a patient with “Class A” predicted hypoglycemic episode, including the CGM (first panel), DR (second panel) and ROC (third panel) traces, for a representative ideal scenario.

FIG. 3 is a graph showing how the proposed method works for a patient with “Class B” predicted hypoglycemic episode, including the CGM (first panel), DR (second panel) and ROC (third panel) traces, for a representative ideal scenario.

FIG. 4 is a graph showing the effects of the proposed method vs the standard protocol, for a subject with “Class B” predicted hypoglycemia, including the CGM trace (first panel, line 20: standard protocol, line 10: proposed method), DR (second panel) and ROC (third panel), in a realistic scenario.

FIG. 5 is a graph showing the effects of the proposed method vs the standard protocol, for a subject with “Class A” predicted hypoglycemia, including the CGM trace (first panel, line 20: standard protocol, line 10: proposed method), DR (second panel) and ROC (third panel), in a realistic scenario.

FIG. 6 includes two graphs showing how the proposed method reduces the time spent in hypoglycemia, with respect to the reference protocol, for 30 subjects with forced hypoglycemic episodes associated to “Class A” (left panel) and “Class B” (right panel).

FIG. 7 includes two graphs showing how the proposed method reduces the post-treatment rebound, with respect to the reference protocol, for 30 subjects with forced hypoglycemic episodes associated to “Class A” (left panel) and “Class B” (right panel).

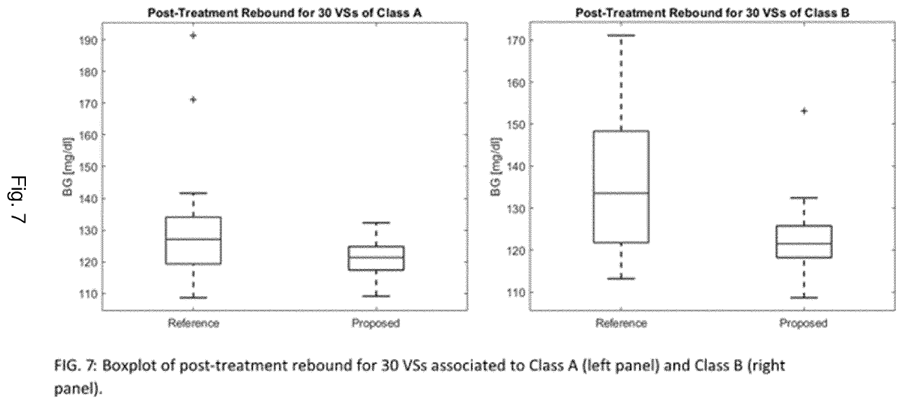

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a”, “an” and “the” includes plural reference, the meaning of “in” includes “in” and “on”.

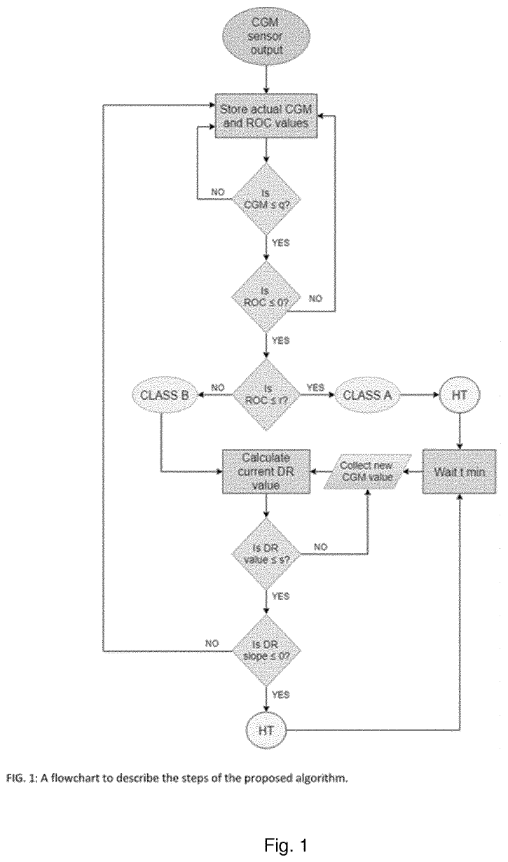

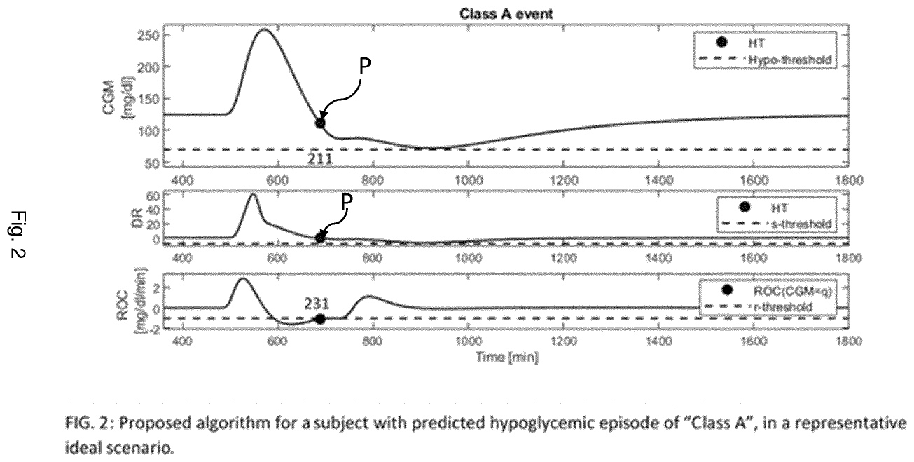

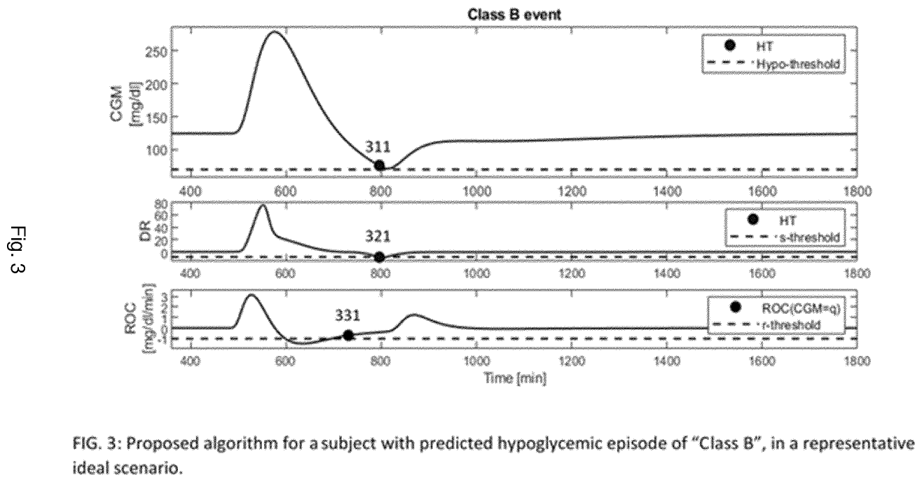

Starting from the flowchart depicted in FIG. 1, which schematizes the procedure to trigger a HT, we present a possible and preferred embodiment. In addition, to fully explain the proposed method, an ideal representative glucose concentration profile is employed. FIGS. 2 and 3 show an example of how the method works in that background, for a preferred embodiment. For both the figures, in the upper panel the glycemic trace with the administered HTs (circles) is depicted. In the middle panel there is the clinical risk trace and in the lower panel there is the glucose ROC. All the thresholds (dashed black lines) will be explained in the following description.
1. The method starts with the real-time collection of the outputs of a CGM sensor, i.e., CGM value and ROC, which the device provides to the user, e.g., every 5 minutes.
2. The method checks for the CGM value until it is near to the hypoglycemic range: if it is lower than or equal to q, the method checks for the ROC value.
3. For positive ROC, the method returns to step 1. If the ROC is negative or equal to 0, the method checks for its value in order to predict the forthcoming hypoglycemic episode and its severity class: patients of “Class B” go in hypoglycemia with a mild ROC, while patients of “Class A” go in hypoglycemia more strongly, i.e., they are supposed to go deeper in the hypoglycemic range without a countermeasure. Therefore, for “Class A”, the HT administration must be much prompter than for “Class B”.
4. If ROC≤r (see FIG. 2 point 231 in the lower panel), the method assigns the hypoglycemic episode to the “Class A” and a first HT administration is triggered (at time 211, see FIG. 2 upper panel). In FIGS. 2 and 3 the dashed black line of the lower panels represents the r threshold. The black circle indicates the moment in which the condition of step 2 is verified. If ROC>r (see FIG. 3 point 331 in the upper panel), the hypoglycemic episode is assigned to the “Class B”.
5. For “Class A” predicted events, the method waits for t min before collecting a new CGM value. For “Class B” predicted events, the method continues to collect CGM values at every sampling time.
6. Every time a new CGM value is collected, the method uses the current output of CGM sensor to compute a clinical risk function. In a preferred embodiment the clinical risk can be measured by the DR concept as follows [8]:

$$D R ( g {,}^{\frac{d g}{dt}} ) = \{ \begin{matrix}S R ( g ) \cdot [ δ \cdot tanh ( α \frac{d r}{dt} + γ ) + β ] & if SR ( g ) > 0 \\ S R ( g ) \cdot [ δ \cdot tanh ( - α \frac{d r}{dt} + γ ) + β ] & if SR ( g ) < 0\end{matrix}$$

with g and

$$\frac{dg}{dt}$$

being the current glycermic reading and its ROC, respectively, α, β, γ being scalars equal to 5, 2.125 and -1.151 and δ being a modulation factor that, in a preferred embodiment, is equal to 1.375. SR(g) is the static risk (SR) function, defined by Kovatchev et al. [10] (Diabetes Care 1997) as:

SR(g)=10·{γ̅·[(ln(g))^{α̅}−β̅]}²
With α̅, β̅ and γ̅ being scalars equal to 1.084, 5.381 and 1.509.
7. For both the classes of events, the method collects a new CGM value until the DR is lower than or equal to s. In this moment it checks for the DR first time derivative.
8. If the DR first time derivative is negative or equal to 0 (see FIG. 3 point 321 in the middle panel) a HT administration is triggered (at time 311, see FIG. 3 point 311 in the upper panel). In FIGS. 2 and 3 the dashed black line of the middle panel represents the s threshold and the circle indicated by reference P corresponds to a HT administered when both the previous conditions verify. After the HT administration, the method waits for t min before collecting new CGM values and suggests a re-treat if all the conditions verify again, in order to prevent a possible relapse in hypoglycemia.
9. If the DR slope is positive, the method returns to the step 1.

In a preferred embodiment, the first step of episode classification (step 2) can be performed when CGM crosses the threshold of q=112.5 mg/dl (which corresponds to DR=SR=0) with negative slope, so the patient's glucose concentration is below target and moving towards hypoglycemia. Administering a HT could be premature for patients who are moving towards hypoglycemia slowly, since it is too early to understand if they will really go below the hypoglycemic threshold (e.g., 70 mg/dl). On the other hand, for patients going more quickly towards hypoglycemia a HT administration is reasonable. Therefore, to classify the severity of patients' about to happen hypoglycemia, the method checks on the ROC value, observing if it is lower or greater than the r threshold. In this preferred embodiment, we set the r threshold r=−1 mg/dl/min.

Generally, to predict the entrance in hypoglycemia by means of the DR, it is possible to exploit its faculty of intrinsically predicting the SR of about 10 min. So, in a preferred embodiment, the value of the scalar parameter s is the value that SR assumes on the hypoglycemic threshold: SR(BG=70 mg/dl)=−7.7550.

Finally, in accordance with 2018 Standards of Medical Care in Diabetes, a possible time interval after which re-check the DR and its derivative, and determine if an additional HT is needed, is t=15 min.

In a preferred embodiment, the HT dose could vary in relationship to the prior on the severity of the predicted hypoglycemic episode: a possible strategy is to fix the first HT at 20 g and set the dose of the subsequent HT at 20 g for “Class A” and 15 g for “Class B” hypoglycemic events.

The presented method has been validated through the Type 1 Diabetes Patient Decision Simulator (T1 D-PDS), developed by Vettoretti et al. [11] (IEEE Trans Biomed Eng 2018): it is a model able to simulate both SMBG measurement and CGM output, including noisy CGM readings (with an error model able to reproduce both the sensor noise and its imperfect calibration step). We tested the methods in silico, since the T1D-PDS allows to test different methods under the same background conditions for each patient. In our case, to evaluate the predictive power of the proposed method, its performance has been compared with that of another protocol, which we call reference protocol, which triggers an HT as soon as the CGM crosses the hypoglycemic threshold, and re-checks patients' glycemia every 15 min, as suggested by ADA 2018 Standards of Medical Care in Diabetes.

Database

The assessment of the method has been performed on 30 virtual subjects (VSs), who have been monitored for 24 h, in a single-meal scenario. The VSs have been forced in hypoglycemia by tuning two parameters: the insulin dose before a meal and the delay in meal bolus administration time. Particularly, two scenarios have been developed, differentiating on the ROC value during the crossing of the hypoglycemic threshold: in “Scenario 1”, the VSs have a ROC between 0 and −1 mg/dl/min, while in “Scenario 2”, the VSs have a ROC between −1 and −2 mg/dl/min. Therefore, the method should be able to associate most of the hypoglycemic episodes of “Scenario 1” VSs to the “Class B”, and those of “Scenario 2” to the Class A.

Metrics

To evaluate the performance of the proposed method, two metrics have been used:
Time spent in hypoglycemia [min], which is the main index of efficiency of the method. It is computed as the time spent under the hypo-threshold of 70 mg/dl;
Post-treatment rebound [mg/dl], which is important to evaluate, in order to avoid a hyperglycemic episode as drawback. It is computed as the maximum BG value after the HT administration.

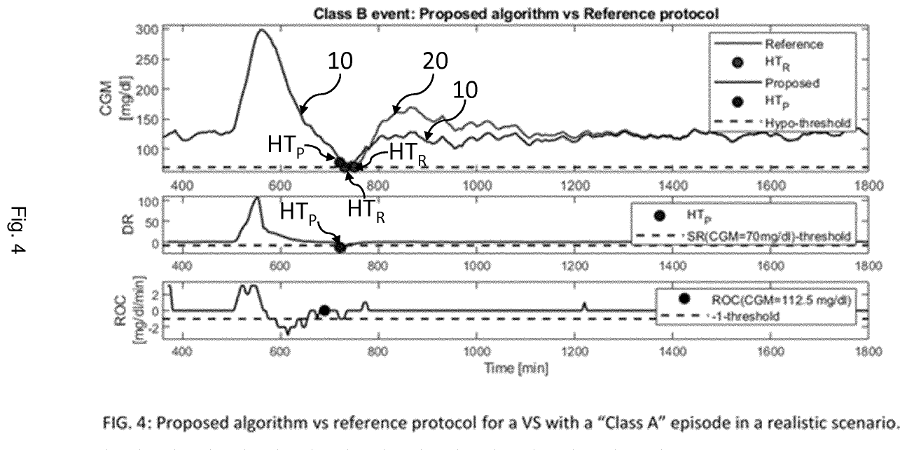

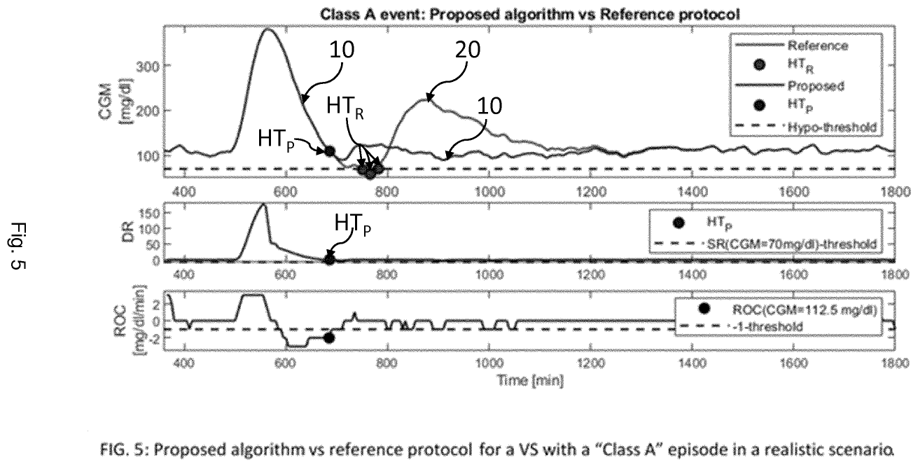

In FIGS. 4 and 5, a comparison between the proposed method (line 10) and the reference protocol (line 20) is presented, for both the classes of predicted hypoglycemic episode.

In FIG. 4, the early HT administration suggested by the proposed method makes one HT sufficient to totally avoid hypoglycemia, while two HT administrations are needed according to the reference protocol, bringing to a higher rebound (upper panel). The administration happens when DR crosses the SR(BG=70 mg/dl) threshold (middle panel). Indeed, when DR=0, the ROC value is not lower than −1 (lower panel) so, according to the classification step rules, it is not necessary to trigger an HT at that moment.

In FIG. 5, the glycemic traces of a VS with a “Class A” hypoglycemic episode are depicted. In the upper panel it is possible to appreciate the further time anticipation of the first HT, which happens, as depicted in the lower panel, when DR=0, since at that moment the ROC value is so low that it is expected a severe hypoglycemic episode. In this way, the VS totally avoids hypoglycemia with only one HT, while adopting the reference protocol, with the same background conditions, three HTs are administered.

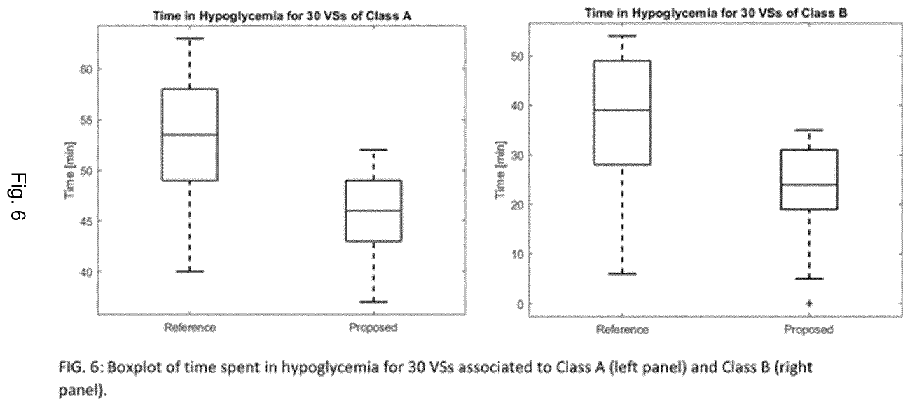

FIG. 6 shows the performance of the proposed method, compared to the reference protocol, in terms of time spent in hypoglycemia in the 30 VSs. There is a substantial reduction of the median time in hypoglycemia, both for “Class A” episodes (left panel) and for “Class B” ones (right panel). This result is supported by a reduction of rebound post-treatment too. So, varying the administration time of the HT, it is possible to obtain a strong reduction both in the time spent in hypoglycemia and in the post-treatment rebound. This result is confirmed by the fact that the mean number of administered HT for the proposed method is reduced compared to a protocol without HT anticipation.

An important issue to be addressed is how the first time BG derivative is computed in the device, since measurement noise can heavily affect the quality of the first derivative signal. If the signal to noise ratio (SNR) is sufficiently high, i.e., the noise has low amplitude compared to the glucose signal, the derivative can be calculated as first order finite differences. If the SNR is low and the noise component is significant, it should be necessary a real-time smoothing of the CGM signal, e.g. obtained via Kalman filter.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

### REFERENCES

[1] American Diabetes Association, Standards of Medical Cares in Diabetes 2018. Diabetes Care. 2018; 41(Suppl. 1):555-64.
[2] Greenburg, A. et al., System for the Detection and Handling of Hypoglycemia. W.O. Patent 2014/140062 A2, Sep. 18, 2014.
[3] Mintz, L. et al., Device and Method for preventing Hypoglycemia. U.S. Patent 2009/0030398 A1, Jan. 29, 2009.
[4] Budiman, E S. et al., Model based variable risk false glucose threshold alarm prevention mechanism. U.S. Patent 2014/0121488 A1, May 1, 2014.
[5] Hughes, C S., Patek, S D., Breton, M D. and Kovatchev B P., Hypoglycemia Prevention via Pump Attenuation and Red-Yellow-Green “Traffic” Lights Using Continuous Glucose Monitoring and Insulin Pump Data. J Diabetes Sci Technol. 2010; 4(5):1146-55.
[6] Kovatchev, B P. et al., CGM-based prevention of hypoglycemia via hypoglycemia risk assessment and smooth reduction of insulin delivery. U.S. Patent 2012/0059353 A1, Mar. 8 2012.
[7] Patek, S D., Magni, L., Dassau, E., Hughes-Karvetski, C., Toffanin, C., De Nicolao, G., Del Favero, S., Breton, M., Dalla Man, C., Renard, E., Zisser, F. J., Doyle III, F J., Cobelli, C., Kovatchev, B P. And International Artificial Pancreas (iAP) Study Group, Modular Closed-Loop Control of Diabetes. IEEE Trans Biomed Eng. 2012; 29(11):2986-99.
[8] Sparacino, G. et al., Alert System for Hypo and Hyperglycemia Prevention Based on Clinical Risk. U.S. Patent 2013/0109944 A1, May 2, 2013.
[9] Guerra, S., Sparacino, G., Facchinetti, A., Schiavon, M., Dalla Man, C. and Cobelli, C., A Dynamic Risk Measure from Continuous Glucose Monitoring Data. Diabetes Technol Ther. 2012; 13(8):843-52.
[10] Kovatchev, B P., Cox, D J., Gonder-Frederick, L A. And Clarke. W., Symmetrization of the Blood Glucose Measurement Scale and Its Applications. Diabetes Care. 1997; 20(11):1655-58.
[11] Vettoretti, M., Facchinetti, A., Sparacino, G. and Cobelli, C., Type-1 Diabetes Patient Decision Simulator for In Silico Testing Safety and Effectiveness of Insulin Treatments. IEEE Trans Biomed Eng. 2018; 65(6):1281-90.

1. A method for continuous monitoring glucose to prevent/mitigate hypoglycemic events in a patient, comprising the steps of:
(a) collecting from a continuous glucose monitoring, CGM, device a periodic series of CGM values and rate of change, ROC, values;
(b) comparing a CGM value to a predetermined threshold q;
(c) if CGM is lower than or equal to q, then comparing the ROC value to zero, else go to step a) (d) if ROC value is lower than or equal to zero, then comparing the ROC value to a predetermined threshold r, else go to step a);
(e) if ROC value is lower than or equal to r, then go to step i);
(f) calculating current Dynamic Risk, DR, value;
(g) if current DR value is lower than or equal to a predetermined s threshold, then calculating the slope of DR function; else go to step k);
(h) if slope of DR function is higher than zero, then go to step a);
(i) alerting about the need of an immediate Hypoglycemic Treatment, HT, administration to the patient;
(j) waiting for a predetermined time interval, t;
(k) collecting a new CGM value and go to step f).

2. The method according to claim 1 wherein the threshold q is equal to 112.5 mg/dl.

3. The method according to one or more of claims 1-2 wherein the threshold r is equal to −1 mg/dl/min.

4. The method according to one or more of claims 1-3 wherein the threshold s is equal to −7.7550.

5. The method according to one or more of claims 1-4 wherein the time interval t is equal to 15 min.

6. The method according to one or more of claims 1-5 wherein DR function is calculated as follows:
$$D R ( g {,}^{\frac{d g}{dt}} ) = \{ \begin{matrix}S R ( g ) \cdot [ δ \cdot tanh ( α \frac{d r}{dt} + γ ) + β ] & if SR ( g ) > 0 \\ S R ( g ) \cdot [ δ \cdot tanh ( - α \frac{d r}{dt} + γ ) + β ] & if SR ( g ) < 0\end{matrix}$$
with g and
$$\frac{d g}{d t}$$
being the current glycemic reading and its ROC, respectively, α, β, γ and δ being scalars; being a modulation factor that, in a preferred embodiment, is equal to 1.375. SR(g) is the static risk (SR) function:
SR(g)=10·{γ̅·[(ln(g))^{α̅}−β̅]}²
α̅, β̅ and γ̅ being scalars.

7. The method according to claim 6 wherein α, β, γ are equal to 5, 2.125 and −1.151 respectively, α̅, β̅ and γ̅ are equal to 1.084, 5.381 and 1.509 respectively and δ is equal to 1.375;

8. A system for continuous monitoring glucose to prevent/mitigate hypoglycemic events in a patient, configured to perform the steps of the method according to any one of claims 1-7, said system comprising:
means for collecting from a continuous glucose monitoring, CGM, device a periodic series of CGM values and rate of change, ROC, values;
means for comparing a CGM value to a predetermined q threshold means for comparing the ROC value to zero and to a predetermined r threshold;
means for calculating current Dynamic Risk, DR, value;
means for comparing DR value to predetermined s threshold;
means for calculating the slope of DR function;
means for alerting about the need of an immediate Hypoglycemic Treatment, HT, administration to the patient.
